(19) United States
(12) Patent Application Publication
(10) Pub. No.: US 2024/0400610 A1
(43) Pub. Date: Dec. 5, 2024

(54) METHOD TO OBTAIN A PURIFIED ANTIBODY COMPOSITION

(71) Applicant: DR. REDDY'S LABORATORIES LIMITED, Hyderabad (IN)

(72) Inventors: Ram KUMAR M, Hosur Krishnagiri District (IN); Roja APUORVA T, Bharathipuram Dharmapuri (IN); Ranjana SINGH, Chapra (IN); Kishore JAHAGIRDAR, Hyderabad (IN)

(21) Appl. No.: 18/687,882

(22) PCT Filed: Sep. 1, 2022
(86) PCT No.: PCT/IN2022/050778

§ 371 Date: Feb. 29, 2024

(30) Foreign Application Priority Data
Sep. 3, 2021 (IN) 2021141040076

(51) Int. Cl. C07K 1/18; C07K 1/22; C07K 16/28

(52) U.S. Cl. CPC C07K 1/18; C07K 1/22; C07K 16/2839

(57) ABSTRACT

The current invention discloses a method for obtaining a purified antibody composition. The method discloses the use of various chromatography steps in a particular order to obtain a purified composition of a therapeutic monoclonal antibody starting from a composition comprising the monoclonal antibody and one or more process and product related impurities. Further, the method also discloses the use of additional purification steps such as depth filtration, diafiltration, ultrafiltration and tangential flow filtration for obtaining the said purified antibody composition.

### FIELD OF THE INVENTION

The present invention relates to protein purification methods. In particular, the invention relates to methods for purifying an antibody composition using chromatography.

### BACKGROUND

Monoclonal antibodies (mAbs) are effective targeted therapeutic agents. The high specificity of the antibodies makes them ideal to reach their intended target and hence they are useful to treat a wide variety of diseases.

The commercial production of recombinant human monoclonal antibody therapeutics demands robust processes, i.e., the purification scheme needs to reliably and predictably produce antibody composition intended for use in humans. A process should be designed to remove the product related contaminants such as high molecular weight (HMW) aggregates, product variants such as charged variants (acidic, deamidated/oxidized, basic), sequence variants and other species, as well as process related contaminants such as leached Protein-A, host cell protein, DNA, adventitious and endogenous viruses, endotoxin, extractable from resins and filters, process buffers and agents such as detergents that may have been employed for virus reduction. In designing a purification scheme and other conditions for each of the chromatographic steps, along with removal of contaminants, an important consideration is recovery from each step of the purification scheme and from the overall purification scheme. Hence, for a commercially viable process, the purification scheme needs to be designed to ensure adequate removal of contaminants from an antibody composition while maintaining the yield of the same.

Product-related and process-related impurities, including aggregates, have the potential to interfere with the purification process, affect the protein during storage, and/or can potentially be a cause of adverse reactions upon administration of an antibody to a subject as a pharmaceutical. Therefore, separation of the desired recombinant therapeutic protein from product- and process-related impurities to a purity sufficient for use as a human therapeutic poses a formidable challenge. This challenge becomes even more daunting in case of a highly hydrophobic antibody, such as an anti-α4β7 antibody, for example, vedolizumab. For a highly hydrophobic antibody such as vedolizumab, Hydrophobic Interaction Chromatography (HIC) is one of the preferred polishing chromatography steps in the art. Although HIC can be an effective step for aggregate clearance and reduction of other process related impurities such as host cell proteins, leached protein-A and endogenous viruses, HIC suffers from the limitation of use of high concentrations of kosmotropic salts to achieve the desired separation. These salts often pose a disposal concern in manufacturing facilities and at times can cause precipitation of the product.

Further to the above requirements, biosimilar manufacturers are additionally tasked with meeting a target quality profile based on the already approved product. A biosimilar candidate needs to not only match the quality profile of the corresponding approved product, but it needs to be cost effective as well, so as to be able to compete with the other biosimilars in the market. This brings in the additional requirement of bringing down costs related to manufacturing, without compromising on the product quality. For a biosimilar manufacturer, the aim is to design a purification scheme which results in a product having pre-defined quality attributes and is economical at the same time.

In light of the above information, there is a need for an improved process, which is cost-effective, to control the product and process related impurities in the final drug substance and obtain a purified antibody composition for use as a human therapeutic. Therefore, the objective of the present invention is to provide a method of purification for obtaining a purified antibody composition of vedolizumab, wherein the process is devoid of HIC.

### SUMMARY

The inventors of the current invention have found that an antibody composition comprising a hydrophobic antibody can by purified to a purity sufficient to be administered as a therapeutic to a human by a process which is devoid of hydrophobic interaction chromatography (HIC). The present invention accordingly discloses a method for obtaining a purified antibody composition comprising a hydrophobic antibody, the method comprising the combination of one or more chromatography steps selected from affinity chromatography, cation exchange chromatography and anion exchange chromatography, wherein the method is devoid of hydrophobic interaction chromatography (HIC).

In some embodiments, the method includes chromatography steps interspersed with additional purification steps including, but not limited to, depth filtration, diafiltration, ultrafiltration, tangential flow filtration and other purification steps well known to a person skilled in the art.

In some embodiments, the method disclosed as per the current invention maintains the level of charge isoforms (including acidic and basic isoforms) of the antibody within a targeted range.

In some embodiments, the method disclosed is a large scale purification method for obtaining purified antibody composition. The method also provides for high recovery of the purified antibody at large scale.

The method disclosed as per the current invention is economically advantageous as it does not require additional polishing chromatographic steps such as HIC or mixed mode chromatography (MMC), which reduces the process time and cost by a significant factor.

### DETAILED DESCRIPTION OF THE INVENTION

### Definitions

The term “contacting” as used herein, refers to applying a solution, e.g., a mixture comprising a protein product and a contaminant, as described herein, to a chromatography matrix. In some embodiments, the term “contacting” is synonymous with “loading” a solution onto a chromatography column. A “chromatography support” as used herein refers to the adsorbent solid material contained within a chromatography column.

The phrase “ion exchange material” refers to a solid phase which is negatively charged (i.e., a cation exchange resin) or positively charged (i.e., an anion exchange resin). The charge may be provided by attaching one or more charged ligands to the solid phase, e.g. by covalent linking. Alternatively, or in addition, the charge may be an inherent property of the solid phase (e.g. as is the case for silica, which has an overall negative charge).

The term “conductivity” refers to the ability of an aqueous solution to conduct an electric current between two electrodes. In solution, the current flows by ion transport. Therefore, with an increasing amount of ions present in the aqueous solution, the solution will have a higher conductivity. The unit of measurement for conductivity is mS/cm, and can be measured using a conductivity meter, e.g., by Orion. The conductivity of a solution may be altered by changing the concentration of ions therein. For example, the concentration of a buffering agent and/or concentration of a salt (e.g. NaCl or KCl) in the solution may be altered in order to achieve the desired conductivity.

A “contaminant” or an “impurity”, as used interchangeably herein, is a material that is different from the desired polypeptide product. The contaminant may be a variant or isoform of the desired polypeptide (e.g. a deamidated variant or an aminoaspartate variant of the desired polypeptide) or another non-product related polypeptide, for e.g., host cell protein, host cell nucleic acid, endotoxin, etc. A contaminant can also be process related, for example—Protein-A-leachates.

The terms “variant” and “isoform” have been used interchangeably throughout the document.

“High molecular weight aggregates” as referred herein encompasses association of at least two molecules of a product of interest, e.g., antibody or any antigen-binding fragment thereof. The association of at least two molecules of a product of interest may arise by any means including, but not limited to, non-covalent interactions such as, e.g., charge-charge, hydrophobic and van der Waals interactions; and covalent interactions such as, e.g., disulfide interaction or non-reducible crosslinking. An aggregate can be a dimer, trimer, tetramer, or a multimer greater than a tetramer, etc.

The term “process or product related impurities” as used herein refer to the contaminants which may be derived from the manufacturing process, for example, but not limited to, cell culture, downstream or cell substrates and may include host cell proteins, host cell DNA, nucleic acid, protein-A leachates etc., or may be molecular variants of the protein of interest, for example, but not limited to, HMW aggregates, acidic variants, basic variants, low molecular weight variants etc., and may be formed during expression, manufacture or storage of the protein.

The term “about” as used herein, would mean and include a variation of up to 20% from the particular value.

The term “large scale” as used herein, refers to manufacturing/producing/obtaining/processing of the desired protein in a bioreactor (or equivalent thereof) of capacity 1000-Liter or more.

The “composition” to be purified herein comprises the protein of interest and one or more contaminants. The composition may be “partially purified” (i.e., having been subjected to one or more purification steps) or may be obtained directly from a host cell or organism producing the antibody (e.g., the composition may comprise harvested cell culture fluid).

The term “Hydrophobic Interaction Chromatography” refers to a form of chromatography that uses a chromatographic support with functional groups that separate proteins on the basis of their hydrophobicity.

The term “Mixed Mode Chromatography” refers to a form of chromatography that uses a chromatographic support with at least two unique types of functional groups, each interacting with the molecule or protein of interest. Mixed mode chromatography generally uses ligands that have more than one type of interaction with target proteins and/or impurities. For example, a charge-charge type of interaction and/or a hydrophobic or hydrophilic type of interaction, or an electroreceptor-donor type interaction. In general, based on the difference in the total interaction, the target protein and one or more impurities can be separated under various conditions.

The term “Anion Exchange Chromatography′” refers to a form of ion-exchange chromatography that uses a support with functional groups that exchange anions.

The term “load” herein refers to the composition loaded onto the chromatography material, i.e., protein-A support, or ion exchange support. Preferably, the chromatography material is equilibrated with an equilibration buffer prior to loading the composition which is to be purified.

The term “bind and elute mode” as used herein refers to a process wherein the target protein substantially binds to the chromatographic support, and is subsequently eluted from the chromatographic support.

The term “flow-through” chromatography, as used herein, refers to a form of chromatography, wherein a chromatography support is used that is not intended to retain or specifically bind with the target protein, e.g., the protein product (e.g, the monoclonal antibody). Under “flow-through” chromatography, a mixture comprising the target protein is applied to the chromatography support, and the effluent comprises the target protein.

Aggregate concentration can be measured in a protein sample using Size Exclusion Chromatography (SEC), a well-known and widely accepted method in the art. Size exclusion chromatography uses a molecular sieving retention mechanism, based on differences in the hydrodynamic radii or differences in size of proteins. Large molecular weight aggregates cannot penetrate or only partially penetrate the pores of the stationary phase. Hence, the larger aggregates elute first and smaller molecules elute later, the order of elution being a function of the size.

### DETAILED DESCRIPTION OF THE EMBODIMENTS

The present invention discloses a method to obtain a purified composition comprising an antibody or antigen binding fragments thereof from a composition comprising the antibody and one or more contaminants, for example, high molecular weight aggregates, host cell proteins/nucleic acids, protein-A leachates, the method comprises the use of a combination of one or more chromatography steps, selected from affinity chromatography, cation exchange chromatography and anion exchange chromatography.

In an embodiment, the method is used to obtain a purified composition comprising an antibody or antigen binding fragments thereof from a composition comprising an antibody and one or more contaminants, the method comprises use of affinity chromatography, cation exchange chromatography and anion exchange chromatography, wherein the affinity chromatography is the first chromatography step.

In some embodiments, the method is used to obtain a purified composition comprising an antibody or antigen binding fragments thereof from a composition comprising an antibody and one or more contaminants, the method comprises use of affinity chromatography in bind and elute mode, cation exchange chromatography in bind and elute mode and anion exchange chromatography in flow-through mode, wherein the affinity chromatography is the first chromatography step.

In some embodiments, the method is used to obtain a purified composition of vedolizumab or antigen binding fragments thereof from a composition comprising vedolizumab and one or more contaminants, the method comprises use of affinity chromatography, cation exchange chromatography and anion exchange chromatography, wherein the affinity chromatography is the first chromatography step.

In some embodiments, the method is used to obtain a purified composition comprising an antibody or antigen binding fragments thereof from a composition comprising an antibody and one or more contaminants, the method comprises use of affinity chromatography, cation exchange chromatography and anion exchange chromatography, wherein the affinity chromatography is the first chromatography step and wherein the method is devoid of hydrophobic interaction chromatography.

In some embodiments, the method is used to obtain a purified composition of vedolizumab or antigen binding fragments thereof from a composition comprising vedolizumab and one or more contaminants, the method comprises use of affinity chromatography, cation exchange chromatography and anion exchange chromatography, wherein the affinity chromatography is the first chromatography step and wherein the method is devoid of hydrophobic interaction chromatography.

In some embodiments, the method is used to obtain a purified composition comprising an antibody or antigen binding fragments thereof from a composition comprising an antibody and one or more contaminants, the method comprises use of affinity chromatography, cation exchange chromatography and anion exchange chromatography, wherein the affinity chromatography is the first chromatography step and wherein the method is devoid of hydrophobic interaction chromatography and mixed mode chromatography.

In some embodiments, the method is used to obtain a purified composition comprising an antibody or antigen binding fragments thereof from a composition comprising an antibody and one or more contaminants, the method comprises use of affinity chromatography, cation exchange chromatography and anion exchange chromatography, in the above order, and wherein the method is devoid of hydrophobic interaction chromatography and mixed mode chromatography.

In any of the above mentioned embodiments, the antibody is an anti-α4β7 antibody or antigen binding fragment thereof.

In some embodiments, the method is used to obtain a purified composition of vedolizumab or antigen binding fragments thereof from a composition comprising vedolizumab and one or more contaminants, the method comprises use of affinity chromatography, cation exchange chromatography and anion exchange chromatography, wherein the affinity chromatography is the first chromatography step and wherein the method is devoid of hydrophobic interaction chromatography and mixed mode chromatography.

In some embodiments, the buffer solutions used at specific pH and conductivity values in AEX and CEX steps leads to the maintenance of the charge isoforms of the antibody in a particular targeted range.

In some embodiments, the method further includes additional purification steps including, but not limited to, viral inactivation, depth filtration, diafiltration, ultrafiltration, tangential flow filtration and other steps well known to a person skilled in the art. These additional purification steps may be interspersed between the chromatography steps.

In some embodiments, anion exchange chromatography is the final chromatography step.

In some embodiments, the purified composition comprises less than 1% HMW aggregates.

In some embodiments, the purified composition comprises less than 0.5% HMW aggregates.

In some embodiments, the purified composition comprises from about 65% to about 75% major isoform. In certain embodiments, the purified composition comprises from about 65% to about 72% major isoform.

In some embodiments, the purified composition comprises less than 16% basic isoforms. In some embodiments, the purified composition comprises 14%-16%, 12%-14%, 10%-12%, 8%-10%, 6%-8% or 4%-6% basic isoforms. In some embodiments, the purified composition comprises 8%-12% basic isoforms. In certain embodiments, the purified composition comprises 9%-11% basic isoforms.

In some embodiments, the purified composition comprises about 15%-25% acidic isoforms. In certain embodiments, the purified composition comprises about 16%-21% acidic isoforms.

In some embodiments, recovery of the antibody from the method disclosed is about 98% or more.

In any of the above mentioned embodiments, the method is also used to reduce the level of other process-related impurities, including but not limited to protein-A leachates, host cell proteins, host cell DNA, etc.

In any of the above mentioned embodiments, the composition comprising the antibody and one or more contaminants may be partially purified or may be obtained directly from cell culture.

In an embodiment, the invention discloses a large scale purification method to obtain a purified composition comprising an antibody or antigen binding fragments thereof from a composition comprising an antibody and one or more contaminants, the method comprises use of affinity chromatography, cation exchange chromatography and anion exchange chromatography, in the above order.

In an embodiment, the invention discloses a large scale purification method to obtain a purified composition comprising an antibody or antigen binding fragments thereof from a composition comprising an antibody and one or more contaminants, the method comprises use of affinity chromatography, cation exchange chromatography and anion exchange chromatography, in the above order and wherein, the method is devoid of hydrophobic interaction chromatography and mixed mode chromatography.

In the above mentioned embodiments, the antibody is an anti-α4β7 antibody or antigen binding fragment thereof.

In the above embodiment, recovery of the anti-α4β7 antibody from the large scale purification method is about 98% or more.

In some embodiments, the anti-α4β7 antibody monomer content from the large scale purification method is about 99.5% or more.

In any of the above mentioned embodiments, the anti-α4β7 antibody is vedolizumab.

The invention is more fully understood by reference to the following examples. These examples should not, however, be construed as limiting the scope of the invention.

### EXAMPLES

### Example 1

### Example 1: Purification Using Affinity Chromatography

A therapeutic monoclonal antibody which binds to human α4β7 integrin, vedolizumab, was cloned and expressed in a Chinese Hamster Ovary cell line and the cell culture broth containing the expressed antibody was harvested and clarified. The clarified cell culture broth was then loaded onto a protein-A chromatography resin (MabSelect™ PrismA) that was pre-equilibrated with a 20 mM phosphate buffer (pH 7.0) containing 150 mM sodium chloride. The column was then washed with the same buffer, followed by a high-salt wash with 200 mM phosphate buffer, 1.5 M NaCl (pH 6.3). The column was then washed with 20 mM phosphate buffer (pH 6.0). The bound protein was eluted using elution buffer containing 124 mM sodium acetate at a pH of 3.4. UV absorbance at 280 nm was measured and the eluate was collected from rising value of absorbance 125 mAU to declining value of absorbance 125 mAU. The eluate from Protein-A affinity chromatography was subjected to low-pH incubation (pH: 3.5±0.2) and depth filtration.

### Example 2: Purification Using Cation Exchange Chromatography

This example describes further purification steps of the therapeutic antibody described in example 1. The eluate from protein-A affinity chromatography was subjected to low-pH incubation and depth filtration, and the filtered liquid comprising the antibody composition was further purified using cation exchange chromatography (CEX). Level of impurities was determined in both load and eluate of CEX. Details of CEX chromatography are given in Tables 1 and 2.

TABLE 1
Chromatography conditions used in CEX

| Resin | Poros ™ XS |
|---|---|
| Bed Height (cm) | 18-22 |
| Residence Time (min) (loading, | 4.0 |
| post-load wash and elution) | |
| Load Factor (g/L) | 40 |

TABLE 2
Details of buffers used in CEX

| | | | Conductivity |
|---|---|---|---|
| Stage | Buffer | pH | (mS/cm) |
| Equilibration | 50 mM phosphate buffer | 5.9 | 4 ± 1 |
| Wash | 50 mM phosphate buffer | 5.9 | 4 ± 1 |
| Elution | Linear gradient, | 5.9 | A: 4 ± 1 |
| | 0%-30% B in 15 CV | | |
| | A: 50 mM phosphate buffer, | | B: 26 ± 2 |
| | B: 50 mM phosphate buffer, | | |
| | 250 mM NaCl | | |
| Regeneration | 0.5M NaOH | NA | NA |

Table 3 summarizes the HMW aggregate level at the time of loading onto CEX and in the eluate obtained from CEX at elution buffer pH of 5.9.

TABLE 3
HMW aggregate level in CEX load and CEX eluate

| | HMW % | HMW % | % | % |
|---|---|---|---|---|
| Batch | (load) | (eluate) | Reduction | Recovery |
| Vmab-1 | 1.5 | 0.3 | 80.0% | 91 |
| Vmab-2 | 1.6 | 0.3 | 81.3% | 94 |
| Vmab-3 | 1.4 | 0.3 | 78.6% | 100 |

Similarly, the levels of HCP and protein-A leachates were determined in CEX load and eluate and are represented in Table 4 along with HCD content in CEX eluate.

TABLE 4
HCP and PAL levels at AEX load and AEX FT stages

| | | |
|---|---|---|
| | | HCD |
| HCP (ng/mg) | PAL (ng/mg) | (pg/mg) |

| Batch | CEX | CEX | CEX | CEX | CEX |
|---|---|---|---|---|---|
| number | load | eluate | load | eluate | eluate |
| Vmab-1 | 170 | 4.4 | 14.0 | 1.6 | 1.3 |
| Vmab-2 | 194 | 4.6 | 16.0 | 2.2 | 0.2 |
| Vmab-3 | 173 | 4.1 | 16.5 | 1.2 | 0.8 |

It is evident from Tables 3 and 4 that the disclosed method is able to significantly reduce the levels of HMW aggregate in addition to the removal of HCP and protein-A leachates.

### Example 3: Purification Using Anion Exchange Chromatography

This example describes the further purification of the therapeutic antibody described in example 1. The eluate from protein-A affinity chromatography was subjected to low-pH incubation and depth filtration, and further polishing steps, including cation exchange chromatography (CEX). The eluate obtained from CEX was subjected to tangential flow filtration (TFF) and the permeate obtained from TFF was used as the load for anion exchange chromatography (AEX). At this stage, HMW aggregate level was determined using analytical size exclusion chromatography and was found to be significantly increased as compared to the level of HMW aggregates in the previous step, i.e., CEX eluate (see Table 7). AEX was then carried out in flow-through mode and the specific pH and conductivity of the loading buffer was used to reduce the level of HMW aggregates. Details of AEX chromatography are given in Tables 5 and 6. It is to be noted that samples Vmab-1 to Vmab-7 were processed at the 50-liter scale and samples Vmab-8 to Vmab-10 were processed at the 1000-liter scale.

TABLE 5
Chromatography conditions used in AEX

| Resin | Q Sepharose FF |
|---|---|
| Bed Height (cm) | 18-22 |
| Residence Time (min) | 6.0 |
| (loading and Post-load wash) | |
| Load Factor (g/L | 150 |

TABLE 6
Details of buffer used in AEX

| | | | Conductivity |
|---|---|---|---|
| Stage | Buffer | pH | (mS/cm) |
| Pre-equilibration | 200 mM phosphate buffer, | 6.30 | 120 ± 5 |
| | 1.5M NaCl | | |
| Equilibration | 35 mM phosphate buffer | 7.0 | 4 ± 1 |
| Post load wash | 35 mM phosphate buffer | 7.0 | 4 ± 1 |
| Regeneration | 0.5M NaOH | NA | NA |

Table 7 shows the HMW aggregate data at the TFF input and outout stages.

TABLE 7
HMW level at TFF stage

| | HMW % | HMW % | % |
|---|---|---|---|
| Batch | (TFF input) | (TFF output) | increase |
| Vmab-1 | 0.3 | 0.6 | 100% |
| Vmab-2 | 0.3 | 0.6 | 100% |
| Vmab-3 | 0.3 | 0.6 | 100% |

The flow-through material obtained from AEX was subjected to analytical SEC to determine the level of HMW aggregate. Table 8 summarizes the HMW aggregate level at the time of loading onto AEX and in the flow-through obtained from AEX.

TABLE 8
HMW aggregate level in AEX load and AEX flow-through stages

| | HMW % | HMW % | % |
|---|---|---|---|
| Batch | (load) | (flow-though) | Reduction |
| Vmab-1 | 0.7 | 0.3 | 57% |
| Vmab-2 | 0.7 | 0.3 | 57% |
| Vmab-3 | 0.6 | 0.3 | 50% |

Similarly, the levels of HCP and protein-A leachates were determined at the AEX load and flow-through stages and are represented in Table 9 along with HCD content in AEX flow-through.

TABLE 9
HCP and PAL levels at AEX load and AEX FT stages

| | | |
|---|---|---|
| | | HCD |
| HCP (ng/mg) | PAL (ng/mg) | (pg/mg) |

| Batch | AEX | AEX | AEX | AEX | AEX |
|---|---|---|---|---|---|
| number | load | FT | load | FT | FT |
| Vmab-1 | 4.50 | 0.40 | 3.10 | 0.10 | 0.5 |
| Vmab-2 | 5.51 | 0.40 | 4.30 | 0.10 | 0.2 |
| Vmab-3 | 4.20 | 0.40 | 3.00 | 0.10 | 0.1 |

It is evident from Tables 8 and 9 that the disclosed method is able to significantly reduce the levels of HMW aggregate in addition to the removal of HCP and protein-A leachates. Further, the pH of the flow-through obtained from AEX chromatography was adjusted to pH 6.0 using 2M acetic acid. TFF was carried out post pH adjustment to concentrate the protein to not less than 70 mg/mL and the TFF retentate was diluted with formulation buffer to prepare the drug substance at a concentration of 65 mg/mL.

The following tables show the level of impurities (charge isoforms, HMW aggregates, HCPs, HCDs, and Protein-A leachates) at the neutralized eluate level, NTEL (post protein-A chromatography stage) and in the drug substance (DS).

Table 10 shows the HMW aggregate level.

TABLE 10
HMW aggregate level at NTEL and DS stage

| | | |
|---|---|---|
| Batch | Monomer (%) | HMW (%) |

| # | NTEL | DS | NTEL | DS |
|---|---|---|---|---|
| Vmab-1 | 98.1 | 99.5 | 1.8 | 0.5 |
| Vmab-2 | 98.0 | 99.5 | 1.9 | 0.4 |
| Vmab-3 | 98.1 | 99.6 | 1.8 | 0.4 |
| Vmab-4 | 98.0 | 99.5 | 2.0 | 0.4 |
| Vmab-5 | 98.0 | 99.2 | 2.0 | 0.6 |
| Vmab-6 | 98.1 | 99.5 | 1.9 | 0.4 |
| Vmab-7 | 98.2 | 99.5 | 1.7 | 0.3 |
| Vmab-8 | 98.7 | 99.6 | 1.2 | 0.2 |
| Vmab-9 | 98.9 | 99.5 | 1.1 | 0.3 |
| Vmab-10 | 98.9 | 99.8 | 1.2 | 0.2 |

Table 11 shows the level of charge isoforms.

TABLE 11
Charge variant level at NTEL and DS stage

| | Acidic | | Major | | Basic | |
|---|---|---|---|---|---|---|
| Batch | isoform (%) | | isoform (%) | | isoform (%) | |
| # | NTEL | DS | NTEL | DS | NTEL | DS |
| Vmab-1 | 21.6 | 17.8 | 67.9 | 65.1 | 10.50 | 10.0 |
| Vmab-2 | 20.1 | 17.2 | 68.9 | 66.9 | 11.00 | 10.2 |
| Vmab-3 | 19.6 | 16.7 | 69.0 | 67.7 | 11.40 | 9.7 |
| Vmab-4 | 16.1 | 17.5 | 69.5 | 72.1 | 14.4 | 10.4 |
| Vmab-5 | 18.2 | 19.4 | 71.5 | 70.5 | 10.3 | 10.1 |
| Vmab-6 | 18.4 | 20.5 | 71.1 | 70.2 | 10.5 | 9.4 |
| Vmab-7 | 18.8 | 20.2 | 71.4 | 70.7 | 9.9 | 9.1 |

Table 12 shows the level of HCPs, HCDs and Protein-A leachates.

TABLE 12
Process related impurities at NTEL and DS stage

| | | | | | Protein-A leachate | |
|---|---|---|---|---|---|---|
| | HCP (ng/mg) | | HCD (pg/mg) | | (ng/mg) | |
| Batch # | NTEL | DS | NTEL | DS | NTEL | DS |
| Vmab-1 | 477 | 0.1 | 89 | 0.1 | 68 | 0.1 |
| Vmab-2 | 516 | 0.1 | 34 | 0.2 | 54 | 0.1 |
| Vmab-3 | 384 | 0.1 | 44 | 0.1 | 68 | 0.1 |
| Vmab-4 | 477 | 1.3 | 217 | 0.2 | 34 | 0.1 |
| Vmab-5 | 677 | 1.1 | 181 | 0.1 | 66 | 0.1 |
| Vmab-6 | 527 | 0.4 | 129 | 0.5 | 52 | 0.1 |
| Vmab-7 | 666 | 0.3 | 117 | 0.1 | 67 | 0.1 |
| Vmab -8 | 563 | <0.4 | 195 | <0.07 | <31.3 | <0.07 |
| Vmab-9 | 525 | <0.4 | 173 | <0.07 | <30.58 | <0.07 |
| Vmab-10 | 498 | <0.4 | 120 | <0.07 | <29.15 | <0.07 |

Table 13 shows the recovery % of the antibody from various process steps.

| | Protein-A | CEX | AEX flow- | |
|---|---|---|---|---|
| Batch | eluate | eluate | through | DS |
| Vmab-8 | 94.4 | 92.9 | 97.4 | 98.0 |
| Vmab-9 | 104.1 | 92.9 | 101.6 | 99.0 |
| Vmab-10 | 99.8 | 94.8 | 98.9 | 100.0 |

1. A method to obtain a purified composition comprising an anti-α4β7 antibody or antigen binding fragments thereof from a composition comprising anti-α4β7 antibody and one or more contaminants, the method comprises
use of affinity chromatography, cation exchange chromatography and anion exchange chromatography, wherein the affinity chromatography is the first chromatography step.

2. The method as claimed in claim 1, wherein the anion exchange chromatography is the final chromatography step.

3. The method as claimed in claim 1, wherein the method is devoid of hydrophobic interaction chromatography.

4. The method as claimed in claim 1, wherein the method is devoid of hydrophobic interaction and mixed mode chromatography.

5. The method as claimed in claim 1, wherein the method is a large scale purification method.

6. The method as claimed in claim 1, wherein the purified anti-α4β7 antibody composition has about 99.5% or more of the antibody in its monomeric form.

7. The method as claimed in claim 1, wherein the anti-α4β7 antibody has a recovery of about 98% or more.

8. The method as claimed in claim 1, wherein the contaminants are selected from high molecular weight aggregates, host cell protein, host cell DNA, protein-A leachates, endotoxins, acidic variants and basic variants.

9. The method as claimed in claim 8, wherein the acidic variants content in the purified composition is about 16%-21%.

10. The method as claimed in claim 8, wherein the basic variants content in the purified composition is about 9%-11%.

11. The method as claimed in claim 3, wherein the hydrophobic interaction chromatography uses a chromatographic support with functional groups that separate proteins based on hydrophobicity.

12. The method as claimed in claim 4, wherein the mixed mode chromatography uses a chromatographic support with at least two types of functional groups.

13. The method as claimed in claim 1, wherein the method further comprises:
subjecting the composition comprising anti-α4β7 antibody to tangential flow filtration.

14. The method as claimed in claim 13, wherein the tangential flow filtration is carried to prior to the anion exchange chromatography.
